SEQUENCE LISTING (32 sequences)

SEQ ID NO: 1 - DNA, 47 bp, Human
caccuacuagaccacuuuuugagccgguuuuuucgggaacuugccaa

SEQ ID NO: 2 - DNA, 50 bp, Human
gaccgguguguccugauccaacugccacaaguaccauaugcaggugacgu

SEQ ID NO: 3 - DNA, 50 bp, Human
gagcgguuaagggagauuuaggcagcagcuuggacaguguaucggcugag

SEQ ID NO: 4 - DNA, 49 bp, Human
gggcgcuuaauguaugccguaugacccucaacauccgacucaguuaagc

SEQ ID NO: 5 - DNA, 48 bp, Human
ccucuugcaccgccgagaauauaauucaagagguccacaacuaauuag

SEQ ID NO: 6 - DNA, 49 bp, Human
ccaagggcuaaguccgcaaauauccuuccuaaaggacucguuacgucgg

SEQ ID NO: 7 - DNA, 42 bp, Human
agaccuuauaccugagauuacacgcucuucgagcacgucgac

SEQ ID NO: 8 - DNA, 50 bp, Human
gcgaaaacuccgauuuuccucuguagugaugggauuuucccgccugaacc

SEQ ID NO: 9 - DNA, 50 bp, Human
caccuaccuaauuauuaaacuuugggcaguaucccgcuuugcuucuuauc

SEQ ID NO: 10 - DNA, 50 bp, Human
guuuauauauacacagguuaagcguaacuucgcuggacagcaagaauccu

SEQ ID NO: 11 - DNA, 50 bp, Human
cauuggccaauuccuugaaucucgacugcucgguagaauagaccuuacca

SEQ ID NO: 12 - DNA, 50 bp, Human
aggagaauuaugagcgggacaacuucguuccguguucgcguacugagcgc

SEQ ID NO: 13 - DNA, 49 bp, Human
cuucucccuugagggccccaugaccugacuguagauaucugcccucgag

SEQ ID NO: 14 - DNA, 50 bp, Human
ugugggccacgcccgauuuuacgcuuuuacccgcacgcgauugguuuguu

SEQ ID NO: 15 - DNA, 50 bp, Human
cagucgucaugguuauagcugccacaaccucgguccugucuucaacggcc

SEQ ID NO: 16 - DNA, 50 bp, Human
gucaagugcacacccuugcucguuucucgaucgccacaaccgauuccaag

SEQ ID NO: 17 - DNA, 50 bp, Human
cuugccguagacccauuuuccaaucacaagucacgcgucucaagcuguua

SEQ ID NO: 18 - DNA, 50 bp, Human
cccguaccaccacacccuaugcacaucguuguuugucgucuuucccgcau

SEQ ID NO: 19 - DNA, 50 bp, Human
aguuucaucguccgagcaagauccuaauggcguccggcgcguuuaugacu

SEQ ID NO: 20 - DNA, 50 bp, Human
cccccauggcacgccgaucacguuuugcuguccgccgguccauaaauacu

SEQ ID NO: 21 - DNA, 49 bp, Human
augacguacccgcacaagccaccacaagucuuaaucgcgccacccuugc

SEQ ID NO: 22 - DNA, 50 bp, Human
acgugcucucaucuuuuaauucgugggcucugcggcuagccucuuagcuc

SEQ ID NO: 23 - DNA, 50 bp, Human
cauuacagcgaaguuaccagccauacacgguacaaaugcgcccgacuagu

SEQ ID NO: 24 - DNA, 49 bp, Human
acggcaacccguuauaaccucccacuggcuaucccguuaagcuucccua

SEQ ID NO: 25 - DNA, 49 bp, Human
ucaccuguacacuaccucuaccaugcuugagccuacgccgccgacaccc

SEQ ID NO: 26 - DNA, 48 bp, Human
cguauucaucagguagcguagauccguguggcgggcuguuccauuuua

SEQ ID NO: 27 - DNA, 50 bp, Human
gccaggguucaucauucacggccgauuucgaagcuccuaacucgagacac

SEQ ID NO: 28 - DNA, 62 bp, Human
  misc_feature (42)..(42): n is a, c, g, t or u
aattaaccctcaaagggaactgttgtgagtctcatgtcgaanttgagcgtctagtcttgtct

SEQ ID NO: 29 - DNA, 44 bp, Human
aattaaccctcactaaagggaactgttgtgagtctcatgtcgaa

SEQ ID NO: 30 - DNA, 40 bp, Human
taatacgactcactatagggagacaagactagacgcrcaa

SEQ ID NO: 31 - DNA, 16 bp, Human
agcagaagacagtggc

SEQ ID NO: 32 - DNA, 19 bp, Human
tagtgcttcctgctgctcc

(19) United States
(12) Patent Application Publication
(10) Pub. No.: US 2007/0066547 A1
(43) Pub. Date: Mar. 22, 2007

(54) Ligands

(76) Inventors: William James, Oxford (GB); Makobetsa Khati, Cape Town (ZA); Jamil Ibrahim, Oxford (GB)

Correspondence Address: PEARL COHEN ZEDEK, LLP;PEARL COHEN ZEDEK LATZER, LLP, 1500 BROADWAY 12TH FLOOR, NEW YORK, NY, 10036, US

(21) Appl. No.: 10/534,259

(22) PCT Filed: Nov. 12, 2003
(86) PCT No.: PCT/GB03/04897

§ 371 Date: Feb. 28, 2006

(30) Foreign Application Priority Data
Nov. 12, 2002 (GB) 0226374.7

(51) Int. Cl. A61K 48/00; C07H 21/04

(52) U.S. Cl. 514/44; 536/23.72

(57) ABSTRACT

Aptamers that bind to viral envelope proteins, in particular those that bind to the envelope glycoprotein gp120 of HIV are disclosed, The use of these aptamers for screening potential therapeutic targets and in the treatment of HIV infections is also described.

The present invention relates to aptamers that bind to viral envelope proteins, more specifically aptamers that bind to the envelope glycoprotein gp120 of HIV.

Viruses can evolve strategies to resist current anti-viral drugs and the selection pressures of humoral and cellular adaptive immunity. For example, HIV-1, R5 strains, which use the CCR5 co-receptor for entry and are the dominant viral phenotype for HIV-1 transmission and AIDS pathogenesis, are relatively resistant to neutralisation by antibodies.

While current anti-viral drugs have prolonged the quality of life for many HIV-1+individuals, they do not eliminate the virus from an infected individual (1, 2). The failure of candidate vaccine antigens based on recombinant HIV-1 surface envelope glycoprotein, gp120, to elicit antibodies that neutralise HIV-1 primary isolates (PIs), and the rapid emergence of drug-resistant HIV-1 strains have encouraged continued efforts to find novel antiretroviral agents with modalities different from those currently in use. One approach is to target the stage at which virus infects host cells. The entry of HIV-1 into target cells, its cellular tropism and the pathogenesis of AIDS are largely determined by the virion surface glycoprotein, gp120, particularly the sequence of the hypervariable loops (3-5). For example, variations in the 10 external portions of the sequence of gp120 determine the cellular tropism of the virus by governing the interaction with chemokine receptors such as CCR5 and CXCR4 (6). Strains that depend on the former co-receptor, known as R5 strains, are preferentially transmitted from host to host (7), dominate the asymptomatic stage of infection (8, 9), and are sufficient to cause AIDS (10).

There are two major phenotypic classes of HIV-1, namely those that infect immortalised lymphoblastoid cells lines preferentially (T-tropic) and those that infect primary macrophages (M-tropic). M-tropic strains are predominant at all stages of infection in vivo and are particularly difficult to neutralise with antibodies.

Aptamers are ligands comprising typically 20 to 120 nucleic acids and can be used to define functionally conserved sites on the surface of proteins. The effect of aptamer-virus binding can be to prevent the infection of cells if the binding site is essential for infection. In the case of HIV, the present drugs on the market all act on intracellular targets, such as reverse transcriptase and protease to prevent replication of the virus. Therefore these drugs can only be used to treat cells that are already infected. As drug-resistant viruses are now appearing it is becoming more important to identify new drugs for antiviral therapy. A treatment that prevented the infection of the cell would be highly desirable. This could be done by targeting the envelope glycoprotein of the virus, with suitable aptamers.

Exemplified aptamers of the present invention are able to bind to the gp120 glycoprotein of a range of strains of HIV-1 and neutralise their infectivity by many orders of magnitude. In the case of the clinically relevant strains that infect only primary leukocytes, the degree of neutralisation we see with aptamers is unprecedented compared with antibodies or any other specific ligand.

Aptamers that bind to the HIV gp-120 molecules have been described previously (Sayer et al (2002) Biochem Biophys Res Commun 293 924-31). However these aptamers were raised against gp120 from a T-tropic strain and were not capable of neutralising the virus. Therefore they are not likely to be useful clinically. The aptamers of the present invention bind to M-tropic gp120, and are capable of neutralising the virus.

Thus in a first aspect the present invention provides a nucleic acid molecule capable of binding to an envelope glycoprotein of an enveloped virus, and neutralising said virus. The virus is preferably HIV, more preferably HIV-1. In one preferred embodiment the glycoprotein is gp120.

In one particular preferred embodiment the aptamer is selected from one of those listed in Table 1.

The term “enveloped” virus is one well known to those skilled in the art and refers to those families of viruses which posses a viral envelope, e.g. retroviruses. Herein, the term “neutralising” refers to neutralising/reducing infectivity of said enveloped virus, preferably by at least one order of magnitude, more preferably by several orders of magnitude.

In a second aspect the present invention provides a method for screening for potential therapeutic targets utilising the aptamers of the invention. As the effect of aptamer-virus binding is to prevent the infection of cells, it is possible to identify small molecules that compete with the aptamer for virus binding. These molecules would bind to the same functionally conserved site on the virus, so inhibit virus infection, and therefore be useful in the development of anti-viral therapeutics. The use of aptamers in high-throughput screens has been described (Green and Janjic (2001) Biotechniques 30 1094-6, 1098, 1100 passim.)

The aptamers of the present invention can themselves be used as therapeutic molecules. Thus in a third aspect the present invention provides a pharmaceutical composition comprising at least one nucleic acid molecule of the invention, optionally together with one or more pharmaceutically acceptable carriers, diluents or excipients.

The nucleic acid can be either RNA or DNA, single or double stranded. Typically the nucleic acid molecules are 20-120 nucleotides in length. The nucleotides that form the nucleic acid can be chemically modified to increase the stability of the molecule, to improve its bioavailability or to confer additional activity on it. For example the pyrimidine bases may be modified at the 6 or 8 positions, and purine bases at the 5 position with CH₃ or halogens such as I, Br or Cl. Modifications of pyrimidines bases also include position 2 modification with NH₃, O⁶—CH₃, N⁶—CH₃ and N²—CH₃. Modifications at the 2′ position are sugar modifications and include typically a NH₂, F or OCH₃ group. Modifications can also include 3′ and 5′ modifications such as capping.

Aptamers can be prepared by methods well known to those skilled in the art, for example by solid phase synthesis (Ogilvie, K. (1988) Proc, Natl, Acad. Sci. U.S.A 85 (16) p 5764-8; Scaringe, S. A (2000) Methods Enzymol 317 p 3-18) or in vitro transcription (Heidenreich, O., W. Peiken and F. Eckstein (1993) Faseb J. 7(1) p 90-6.)

The compositions of the invention may be presented in unit dose forms containing a predetermined amount of each active ingredient per dose. Such a unit may be adapted to provide 5-100 mg/day of the compound, preferably either 5-15 mg/day, 10-30 mg/day, 25-50 mg/day 40-80 mg/day or 60-100 mg/day. For compounds of formula I, doses in the range 100-1000 mg/day are provided, preferably either 100-400 mg/day, 300-600 mg/day or 500-1000 mg/day. Such doses can be provided in a single dose or as a number of discrete doses. The ultimate dose will of course depend on the condition being treated, the route of administration and the age, weight and condition of the patient and will be at the doctor's discretion.

The compositions of the invention may be adapted for administration by any appropriate route, for example by the oral (including buccal or sublingual), rectal, nasal, topical (including buccal, sublingual or transdermal), vaginal or parenteral (including subcutaneous, intramuscular, intravenous, intrathecal, intraocular, or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions. Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils. For applications to the eye or other external tissues, for example the mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administration to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists which may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets.

Preferred unit dosage formulations are those containing a daily dose or sub-dose, as herein above recited, or an appropriate fraction thereof, of an active ingredient.

It should be understood that in addition to the ingredients particularly mentioned above, the formulations may also include other agents conventional in the art having regard to the type of formulation in question, for example those suitable for oral administration may include flavouring agents. In an additional aspect the present invention provides:
(i) the use of at least one nucleic acid molecule of the present invention in the manufacture of a medicament for use in the treatment of HIV infection; and
(ii) a method for the treatment of HIV infection comprising administering an effective amount of at least one nucleic acid molecule of the invention to a subject.

The present invention will now be described in more detail with reference to the following examples, which should not be construed as in any way limiting the scope of the invention.

### THE EXAMPLES REFER TO THE FIGURES

FIG. 1: Isolation of aptamers that bind the gp120 of an R5 tropic HIV-1_{Ba-L}. (A) After four rounds of selection, the polyclonal pool of 2° F.—RNA showed strong and specific binding to HIV-1_{Ba-L}gp120, and binding was enriched by a further selection cycle. (B) Aptamer B19 bound gp120 from both HIV-1_{Ba-L}and HIV-1_{IIIB}. An aptamer concentration of 100 nM was used in all the experiments.

FIG. 2: Neutralisation of R5 HIV-1 strains by monoclonal aptamers. Virus was titrated by limiting dilution in PBMC in the presence of the 100 nM of the aptamers indicated. No reduction infectivity is indicated by open columns, between 10 and 100—fold reduction of infectivity by shaded columns, 10³-10⁴—fold reduction of infectivity by hatched columns and >10⁴—fold reduction of infectivity by solid columns. A and B show neutralisation of HIV-1_{Ba-L}, the strain from which the aptamers were raised. Seventeen aptamers neutralised the virus by 3-4 log₁₀ IU/ml, and two aptamers B4 and B84 inhibited HIV-1_{Ba-L} entry by more than 4 log₁₀ IU/ml. (C) Aptamer B4 inhibited HIV-1_{Ba-L} entry in a concentration-dependent manner, and with an IC₅₀ value of less than 1 nM. (D) Five of the aptamers tested so far also cross-neutralised another R5 strain, HIV-1_{ADA}.

FIG. 3: Failure of HIV-1_{Ba-L} to escape neutralisation by aptamer B4. (A) The predicted sequence of gp120 deduced from sequencing env amplified from four break-through clones of virus following five rounds of selection and passage of HIV-1_{Ba-L} in PBMC in the presence of aptamer B4. The sequences are aligned to that of the inoculum virus (NIH510*) and the database sequence of HIV-1_{Ba-L} (M68893). Dashes represent unsequenced portions. “X” indicates an uncertain residue. Dots indicate identity with the database sequence. (B) Complete neutralisation of clonal HIV-1_{Ba-L}—derived breakthrough stock with 100 nM of aptamer B4 in human PBMC as assayed by the TCID₅₀. (C) Graphical depiction of the TCID₅₀ data as statistically analysed with the ID₅₀ software, showing neutralisation of HIV-1_{Ba-L} breakthrough clone by more than 4 log₁₀ IU/ml, compared to control aptamer. The results are representative of all the clones in that all the four break-through clones were equally neutralised.

FIG. 4: Effect of aptamer B4 on the binding of mAbs to gp120. Recombinant Ba-L gp120 was captured on the surface of a microtitre plate by polyclonal antibody. If indicated, the bound gp120 was then allowed to interact with soluble CD4. The bound gp120 or gp120/CD4 complex was then incubated with varying concentrations of aptamer B4 in triplicate. The gp120 aptamer or gp120/CD4/aptamer complexes were then incubated with the anti-gp120 mAbs indicated at a concentration previously determined to be in the linear detection range of each. Bound mAb was then detected using an anti-Ig-HRP system. The results are displayed as a % reduction from the aptamer-free control value ± standard error.

### EXAMPLE

Materials and Methods

Virus Stocks

All HIV-1 strains used in this study were obtained through the AIDS Research and Reference Reagent Program, NIAD, NIH, Bethesda, USA. HIV-1_{Ba-L} was contributed by S. Gartner, M Popovic and R. Gallo (19), HIV-1_{ADA} by H. Gendelman (20), and HIV-1_{IIIB} by R. Gallo (21)

Monoclonal Antibodies

Anti-gp120 monoclonal antibodies 17b (22), & 48d (23) 2G12 (24) IgG1 b12 (25), and polyclonal human HIV-Ig (26) were obtained from the NIH AIDS Reagent Program (www.aidsreagent.org). mAb 19b was kindly provided by James Robinson, Department of Pediatrics, University of Connecticut, Farmington, Conn., 06030, USA. Anti-gp120-CD4 complex monoclonal antibody CG10 (27) and recombinant CD4-Ig were obtained from the NIBSC Centralised Facility for AIDS Reagents. Anti-FLAG M2 and anti-mouse IgG HRP monoclonal antibodies were obtained from Sigma.

Cells

Spodoptera frugiperda Sf9s cells were kindly provided by Ian Jones (Reading University, UK). Human leukocytes were obtained from buffy coat fractions supplied by Bristol Hospital Services, through the Oxford National Blood Services.

Oligonucleotides (listed 5′-3′) The “Library” oligonucleotide had the composition,

AATTAACCCTCACTAAAGGGAACTGTTGTGAGTCTCATGTCGAA
(N)₄₉TTGAGCGTCTAGTCTTGTCT.
“5′ primer” was:
AATTAACCCTCACTAAAGGGAACTGTTGTGAGTCTCATGTCGAA
“3′ primer” was:
TAATACGACTCACTATAGGGAGACAAGACTAGACGCTCAA.
“Env₆₃₀₉+” primer was
AGGAGAAGAGAGTGGC.
“Env₈₀₂₃−” primer was
TAGTGCTTCCTGCTGCTCC.

Expression of HIV-1_{Ba-L} g120

Sf9s cells were cultured at 28° C., in SF 900 II serum-free insect medium (GibcoBRL) in suspension culture below 1×10⁶ cell/ml. Sf9s cells were transfected with a mixture of 500 ng p 2BaC-gp120 (28) encoding HIV-1_{Ba-L} SU glycoprotein (gp120) and linearised pAcBAK6 (Invitrogen) to generate recombinant virus following standard methods (29). Cells were infected at an m.o.i. of 5 and incubated for 4 days at 28° C., at which time secretion of gp120 into the medium was optimal. gp120 was purified from clarified culture supernatants using anti-FLAG M2 (Sigma) affinity chromatography and fractions were evaluated by SDS-PAGE and western blotting. Protein was further purified by FPLC gel filtration using Superdex 200 HR10/30 (Pharmacia) to exclude high order aggregates and quantified using BCA protein assay kit (Pierce, Chester, UK) according to manufacturer's instructions.

In Vitro Transcription

225 pmol of DNA template was added to a final 500 μl transcription reaction comprising 1 mM 2′F UTP, 1 mM 2′F CTP (TriLink, USA), 1 mM GTP, 1 mM ATP (Amersham-Pharmacia), 40 mM Tris-Cl, pH 7.5, 6 mM MgCl2, 5 mM DTT, 1 mM spermidine and 1500 units of T7 RNA 15 polymerase (New England BioLabs) and incubated at 37° C. for 16 h. Transcription was terminated by addition of 1 unit RNase-free DNase I (Sigma) per ng of DNA template used, and the reaction was incubated for 15-30 min. at 37° C., followed by phenol/chloroform extraction. The RNA was precipitated using ethanol, redissolved in water, separated from low Mr contaminants using a Sephadex-G50 nick spin column (Pharmacia-Amersham) and quantified by determination of A₂₆₀. RNA was refolded by heating in water to 95° C. for 3 min and then cooling to room temperature for 10 min, at which temperature ⅕ volume of 5× HBS buffer (final concentration, 10 mM HEPES, pH 7.4, 150 mM NaCl, 1 mM CaCl2, 1 mM MgCl2, 2.7 mM KCl) was added, incubation continued at room temperature for 5 min.

In Vitro Selection of Aptamers

A BIAcore (Stevenage, UK) 2000 biosensor instrument was used. 20 000 RU of HIV-1_{Ba-L} gp120 was directly coupled to the carboxymethylated dextran surface of CM5 biosensor chips (research grade; BIAcore) using amine coupling through lysine residues following a standard protocol (30). RNA was prepared as described above in HBS. For the first round of selection, 20 μg of the RNA pool (a theoretical diversity of =1014 molecules) was injected at 1 μl/min at 37° C. over the flow cell in which the gp120 was immobilised. Non-specifically bound RNA was removed by injecting 100 μl of the HBS buffer at 5 μl/min. Bound RNA was eluted with 100 μl of 7 M urea at 5 μl/min, and was deproteinised by extraction with phenol/chloroform and then precipitated with ethanol. Recovered RNA was reverse-transcribed to cDNA and PCR— amplified using the 3′ and 5′ primers described under slightly mutagenic conditions. The RNA: protein ratio was increased by a factor of about 4 each round to increase the stringency of selection. At every selection round the RNA was pre-cleared against at least two uncoupled sensor chip flow cells to serve as controls, and also to avoid the inadvertent selection of aptamers that might bind the chip matrix.

Analysis of Aptamer-gp120 Interaction by SPR Biosensor

Affinity measurements were performed at 37° C. 5000-7500 RU of HIV-1 gp120 was covalently immobilised on the chip as described above. Aptamer or control nucleic acids were prepared at a range of concentrations (5 nM-3 200 nM), injected at 5 μl/min (KININJECT procedure) and allowed to dissociate over 60 min. The ligand was regenerated by injecting 1-5 μl of freshly 20 prepared 100 mM NaOH to dissociate any RNA that was still bound without affecting the ability of gp120 to bind soluble CD4. Data were analysed using the BIAevaluation 3.0 (BIAcore) and GraphPad Prism 3.00 (GraphPad software Inc., USA) and the K_{D} was calculated from the ratio of k_{off} and kₒₙ.

Cultivation of Human Peripheral Blood Mononuclear Cells (PBMC)

These were isolated by Ficoll-Hypaque (Pharmacia-Amersham, UK) density gradient centrifugation from heparinised buffy coats of normal, HIV-negative donors. The diluted, autologous plasma was saved, heat-inactivated and clarified to provide autologous serum (AS) supplement for leukocyte culture. The PBMC were washed 6 times in PBS (Sigma) at 4° C. and were essentially free of platelets and granulocytes. PBMC cultures were cultivated without mitogen activation and without the addition of specific growth factors such as IL-2, and were maintained in X-VIVO-10 (Bio-Whittaker) containing 2% AS. This procedure produces a slowly proliferating mixed culture of lymphocytes and macrophages that in our hands supports a higher level of replication of primary isolates than mitogen-treated, cytokine-supplemented cultures.

Virus Infectivity and Neutralisation Assays

Day 7 PBMC were infected with serially diluted virus that had been incubated with 100 nM of anti-gp120 monoclonal aptamer or control aptamer, SA19 (17). Eight replicates were used at each 10-fold dilution. After 16 hours post infection the medium containing virus inoculum and aptamer was replaced with fresh culture medium. Cultures were maintained for 14 days before preparing DNA for LTR-PCR as described previously (31).

Selection for Aptamer Escape Mutants

To attempt to generate aptamer B4 break-through variants, day 7 human PBMC (107) were infected with HIV-lBa-L (105 IU), previously incubated with aptamer B4 (1 nM). Samples of cell supernatant were collected every other day, until cytopathic effects were exhibited. The sample with the highest infectious titer was used to infect freshly differentiated PBMC in the absence of aptamer, in order to amplify virus to approximately 105 IU/ml. Four further cycles of selection and amplification were done following the same protocol, each selection using increasing concentrations of aptamer B4 (i.e. 5; 15; 50 and 100 nM). After the final round of selection, virus was cloned by limiting dilution in fresh PBMC. The env gene was PCR— amplified from virus-positive wells using the Env₆₃₀₉+ and Envs₈₀₂₃− primer pair and the high fidelity PCR system kit (Roche, Germany). The env gene of original seed HIV-1_{Ba-L} virus was similarly obtained.

Binding Site Mapping by Antibody Inhibition

This was done by competitive ELISA largely as previously described (32) with modifications as follows. Briefly, gp120 was captured in an Immulon II ELISA plate (Dynatech Ltd) using D7324 anti-gp120 COOH peptide antiserum (Aalto Bioreagents Plc.) After washing, bound gp120 was incubated with either 50 μl of HBS buffer or 10 μM soluble human CD4 in HBS buffer for 1 hour at room temperature. The plate was washed, and 50 μl of aptamer B4 was added at a range of concentrations, in triplicate, in HBS binding buffer for 1 hour. Anti-gp120 mAbs were added at a concentration previously determined to be within the linear range. After washing, bound antibody was detected using the ABC Elite amplification kit (Vector).

Results

Selection of aptamers against HIV-1_{Ba-L} gp120

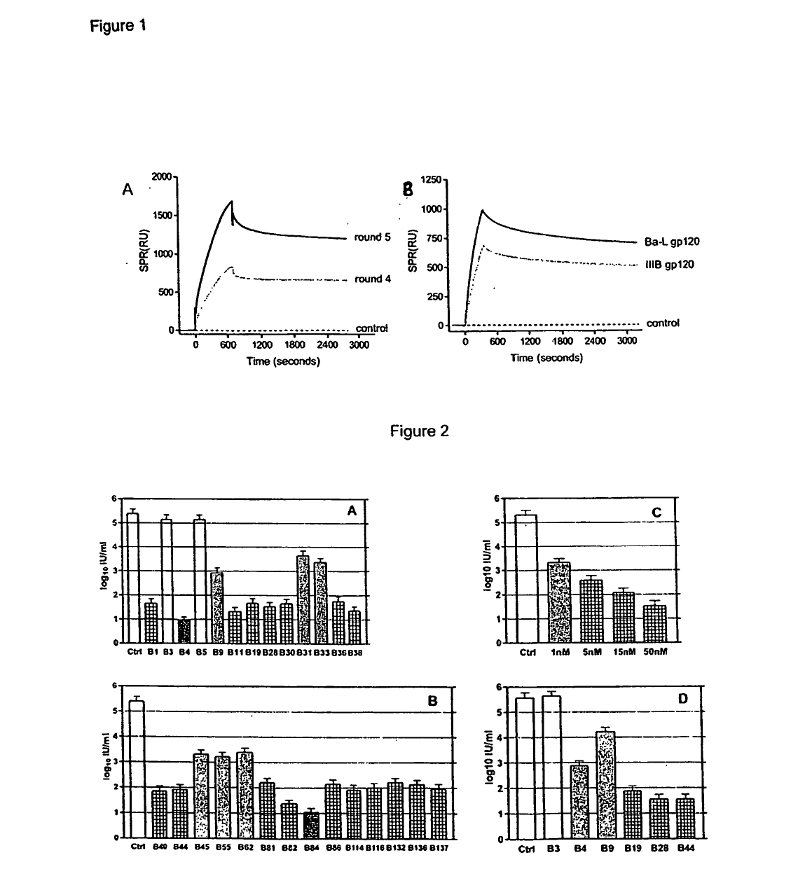

We selected 2′-fluoropyrimidine-containing RNA (2′-F—RNA) aptamers against the gp120 of the R5 strain, HIV-1_{Ba-L}. The target protein was produced as previously described (28) and selection of aptamers was done using a modification of the SELEX protocol (33, 34) in which the target was immobilised on a BIAcore biosensor chip and enrichment was based on the slow dissociation rate of aptamers from target (FIG. 1A). We used 2′-F-RNA aptamers because they are not only resistant to nucleases, but also widen the spectrum of potential tertiary conformations (16), and give rise to more compact and rigid ligands with higher affinities compared to unmodified RNA or NH2-subsituted RNA aptamers (35).

Immobilised gp120 initially bound less than 0.1% of the applied RNA, but this rose to 1.5% at round 2, 16% at round 3, 60% at round 4 and 75% at round 5. The PCR amplified DNA pool from the fifth round of selection was TA cloned. Each clone was screened by BIAcore analysis and those that bound the gp120 with high affinity (Kd 5-100 nM) as determined by BIAcore analysis, were found to belong to at least 25 distinct sequence families (Table 1). Although most of the monoclonal aptamers bound selectively to HIV-1_{Ba-L} gp120, some, such as B19, showed significant binding to the gp120 derived from HIV-1_{IIIB}, an X4 virus (FIG. 1C). Taken together these data imply that a large number of different sequences can fold to give gp120-binding aptamers, and that some of these aptamers might recognise a site on gp120 that is structurally conserved between these two R5 and X4 strains. Neutralisation of HIV-1 by Aptamers Antibodies elicited during natural infection with HIV-1 are generally poorly neutralising, and are generated late in infection (36). Moreover, monoclonal antibodies (mAbs) that recognise epitopes presented in the form of isolated gp120 usually do not bind the same epitope in the context of the assembled virion (37). This underlies the relative resistance of HIV-1 to neutralisation by mAbs, which is particularly pronounced in primary isolates. In this study we therefore asked whether the aptamers isolated against HIV-1_{Ba-L} gp120 could prevent or limit HIV-1 infectivity of target cells. Using an end-point dilution and PCR-based TCID50 assay (31, 38), we showed that 25 of the 27 aptamer clones assayed neutralised homologous HIV-1_{Ba-L} in PBMC (FIGS. 2A and B). Most of these aptamers neutralised HIV-1_{Ba-L} by more than 1000-fold, and one aptamer (B4) neutralised the virus by about 5 log₁₀. B4 inhibited HIV-1_{Ba-L} entry with a 50% inhibition concentration (IC50) value of less than 1 nM in human PBMC (FIG. 2C). All HIV-1_{Ba-L} neutralising aptamers tested so far, including B4, also cross-neutralised another R5 strain, HIV-1_{ADA}, (FIG. 2D). The gp120 of these two strains is only 84% identical and they show differences in the degree of their macrophage tropism (38). Therefore the ability of at least five aptamers to neutralise at least two HIV-1 strains suggests that they might recognise functionally important sites on gp120 that are conserved between at least the R5 members of this clade B sub-type.

HIV-1_{Ba-L} does not Mutate to Escape Neutralisation by Aptamer B4

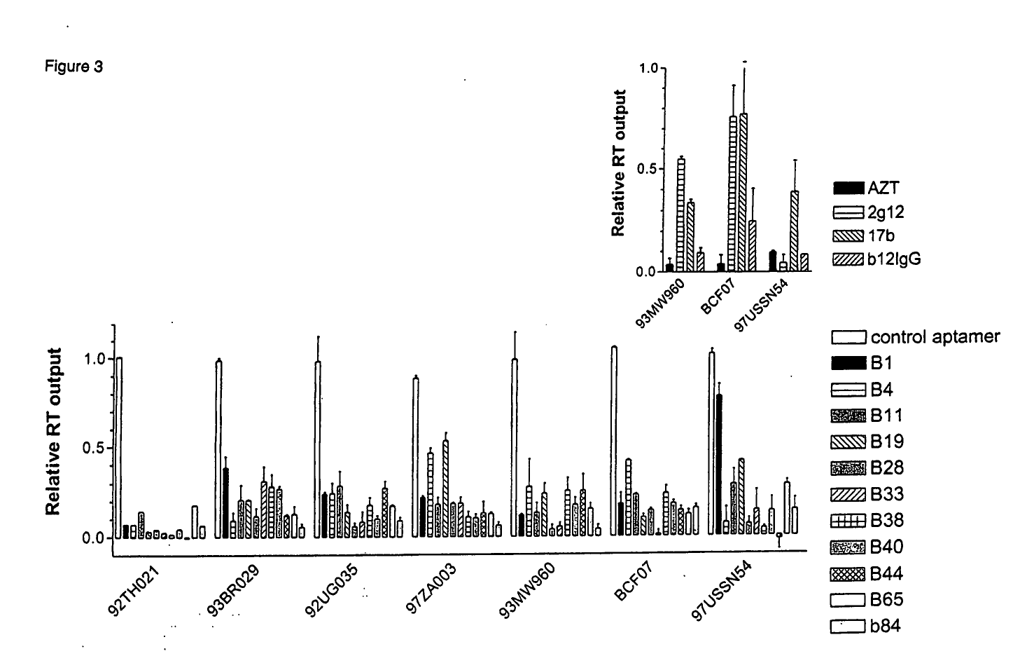

HIV-1 mutates readily during infection in vivo in response to selection pressures, thereby propagating variants that can escape immune recognition and become resistant to anti-viral drugs (39). It has recently been shown that mutants of HIV-1 can resist the inhibitory effects of a CCR5 antagonist without resorting to the use of alternative co-receptors (40) and the possibility that similar escape variants might have increased virulence raises concerns about the use of such drugs (41). We therefore tested whether HIV-1_{Ba-L} could mutate to escape neutralisation by aptamer B4 by selecting for variants that could grow in human PBMC after challenge with the aptamer, as described in the Methods. Following selection through five cycles of progressively increasing aptamer concentration, four break-through clones of virus were isolated. The gp120-encoding portion of the env gene was determined for each clone and 25 compared with that of the parental virus (FIG. 3A). We observed that the parental sequence diverges from that in the database by 1.9%, though it is more closely related to that of the official Ba-L sequence than to that of any other HIV-1 strain in the database (analysis not shown). All four break-through clones had several additional amino acid substitutions in gp120, including in the putative neutralisation epitope at the V3 loop tip. We then tested whether these mutations had conferred resistance to aptamer B4 neutralisation by expanding the HIV-1_{Ba-L}—derived clones, followed by re-challenging the expanded virus with aptamer B4 and infection of human PBMC. Aptamer B4 neutralised all the break-through clones by up to 10⁵-fold (FIGS. 3B and 3C), implying that the mutations did not confer any selective advantage to HIV-1_{Ba-L}. These data suggest that aptamer B4 might bind to a region of the gp120 that is critical to HIV-1_{Ba-L} function and therefore unable to mutate without detrimental effects to the virus.

Binding of Aptamer B4 to gp120 Affects the Conserved CCR5-Binding Surface

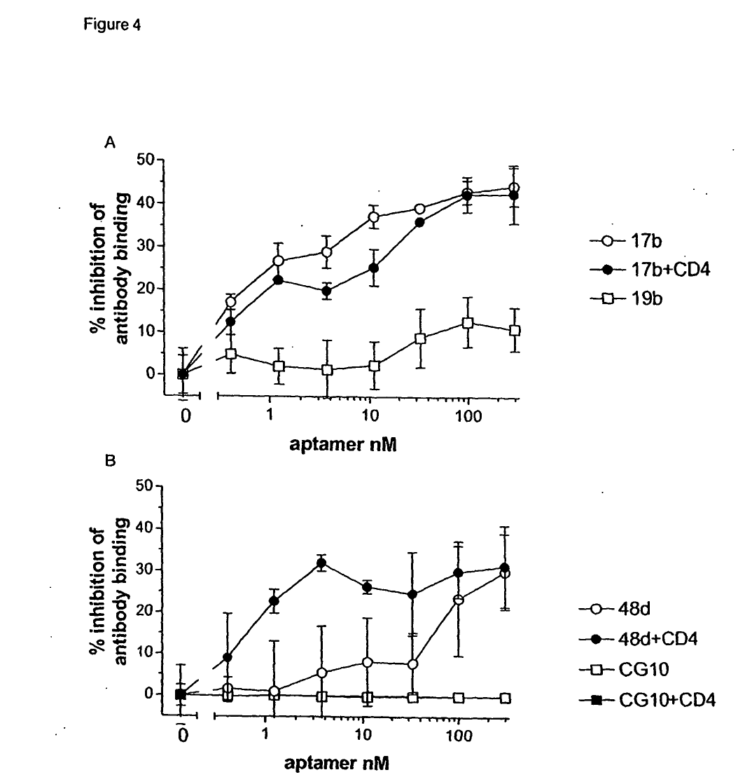

A likely mechanism of neutralisation by aptamer was by inhibition of virus-receptor interaction, however, using SPR biosensor analysis, we found that B4 did not interfere with the binding of sCD4 to monomeric, immobilised gp120. We next tested whether B4 would interfere with the binding of monoclonal antibodies whose epitopes had been previously mapped on gp120. The mAbs we chose were all neutralising as we hypothesised that B4 was likely to bind close to a conserved neutralisation epitope, rather than to a non-neutralising epitope. Of the ten mAbs tested, only one, 17b, was convincingly inhibited by aptamer in the absence of CD4 (see FIG. 4A). The level of inhibition was significant but only 50% at the highest concentration of aptamer, suggesting that either the two molecules bound to spatially related but distinct surfaces or that the binding of B4 produced a subtle allosteric change in the epitope of 17b, reducing the level of antibody binding without abolishing it. The 17b epitope is partially obscured from antibody in the absence of CD4-binding by the V1 and V2 hypervariable loops of gp120 (23, 42) and overlaps with the binding site for the virus' major co-receptors, CCR5 and CXCR4 (43). The binding of a second antibody, 48d, which like 17b maps to the “CD4i” epitope, was inhibited by high concentrations of aptamer in the absence of CD4 but by nanomolar concentrations in the presence of CD4 (see FIG. 4b). A third CD4i-binding antibody, CG10, was not inhibited by aptamer B4 at all.

Discussion We present a novel strategy for identifying conserved regions of the envelope of primary HIV-1 isolates that could be targeted by potentially therapeutic agents. The small size and biophysical properties of aptamers have enabled us to target conserved sites on HIV-1 gp120 whose ligation produced highly efficient neutralisation of infectivity. Studies on the structure of gp120 complexes derived from HIV strains of differing co-receptor utilisation indicate that, although the surface loops of gp120 are of variable sequence and variable topology, the core of the protein, containing the principal receptor-interacting surfaces, is remarkably conserved in tertiary structure (12). Quaternary interactions between variable surface loops of gp120 monomers seem to have evolved to enable the virus to escape from neutralisation by antibody but, critically, expose the conserved core of gp120 to interference by smaller ligands, such as aptamers.

Two properties of aptamer neutralisation deserve particular comment: potency and resistance to escape. The ability of an aptamer like B4 to reduce the infectivity of R5 virus is remarkable when compared with the most potent antibodies, such as IgG1-b12 and 2G12. These antibodies typically reduce infectivity of R5 strains by a single order of magnitude at concentrations of approximately 300 nM (44) whereas, at 100 nM, aptamer B4 reduces infectivity by more than four orders of magnitude. Even at the highest concentrations practicable, specific neutralisation of PI HIV-1 by antibody rarely exceeds two orders of magnitude. Secondly, antibody-mediated neutralisation is rapidly overcome by virus evolution both in vitro (45) and in vivo (46).

Other therapeutic strategies designed to interfere with HIV-entry include gp41-disrupting peptide analogs (47-49) and small-molecule ligands of CCR5 (40). The most promising, such as C34 (50) and T-20 (51) have IC50 values almost comparable to the aptamers described here (˜2 nM). However, virus variants, resistant to inhibition by T-20 peptide, have now been identified in clinical trials (52).

Our results give some clues to the mechanism of neutralisation by aptamer. Aptamer B4 did not interfere with the interaction between CD4 and gp120, nor the binding of antibodies whose epitopes on gp120 comprise the V3 loop, the V1V2 loops and the carbohydrate on the “silent” face. The ability of potently neutralising aptamer B4 to partially inhibit binding of mAbs 17b and 48d suggests that the mechanism of neutralisation may be by interfering with the interaction of gp120 with its co-receptor, CCR5. In the absence of binding of CD4 to gp120, the 17b/48d epitopes are partially occluded on monomeric gp120 and fully occluded on functional, trimeric gp120, ensuring that it is only exposed to the antiviral effects of the humoral immune response very transiently during infection, if at all. In contrast, perhaps because of its smaller size or biophysical properties, aptamer B4 binds to its neutralisation site in the absence of CD4 binding. The CD4-dependent binding of antibody 48d to gp120 was more potently inhibited by aptamer B4 in the presence of CD4, implying that the access of aptamer B4 to the 48d epitope may be partially blocked on uncomplexed gp120. Intriguingly, we found that CG10, a third antibody that maps to the “CD4i” region, was not inhibited by aptamer B4 at all. This is consistent with the location of its epitope closest to the base of the V1V2 loops, and therefore the most occluded of these three epitopes (15, 53). Taken together with the evidence that the B4 binding site is highly conserved, this suggests that, as we had hoped, the aptamer approach has identified a more circumscribed and functionally important neutralisation target on gp120 than has been possible previously using antibodies.

### REFERENCES

1. Finzi, D., Blankson, J., Siliciano, J. D., Margolick, J. B., Chadwick, K., Pierson, T., Smith, K., Lisziewicz, J., Lori, F., Flexner, C., Quinn, T. C., Chaisson, R. E., Rosenberg, E., Walker, B., Gange, S., Gallant, J. & Siliciano, R. F. (1999) Nat Med 5,512-7.
2. Piot, P., Bartos, M., Ghys, P. D., Walker, N. & Schwartlander, B. (2001) Nature 410, 968-73.
3. Chesebro, B., Nishio, J., Perryman, S., Cann, A., O'Brien, W., Chen, I. S. & Wehrly, K. (1991) J Virol 65, 5782-9.
4. Mondor, I., Moulard, M., Ugolini, S., Klasse, P. J., Hoxie, J., Amara, A., Delaunay, T., Wyatt, R., Sodroski, J. & Sattentau, Q. J. (1998) Virology 248, 394-405.
5. Chavda, S. C., Griffin, P., Han-Liu, Z., Keys, B., Vekony, M. A. & Cann, A. J. (1994) J Gen Virol 75 (Pt 11), 3249-53.
6. Berger, E. A., Doms, R. W., Fenyo, E. M., Korber, B. T., Littman, D. R., Moore, J. P., Sattentau, Q. J., Schuitemaker, H., Sodroski, J. & Weiss, R. A. (1998) Nature 391, 240.
7. Samson, M., Libert, F., Doranz, B. J., Rucker, J., Liesnard, C., Farber, C. M., Saragosti, S., Lapoumeroulie, C., Cognaux, J., Forceille, C., Muyldermans, G., Verhofstede, C., Burtonboy, G., Georges, M., Imai, T., Rana, S., Yi, Y., Smyth, R. J., Collman, R. G., Doms, R. W., Vassart, G. & Parmentier, M. (1996) Nature 382, 722-5.
8. Gendelman, H. E., Orenstein, J. M., Baca, L. M., Weiser, B., Burger, H., Kalter, D. C. & Meltzer, M. S. (1989) Aids 3, 475-95.
9. Ping, L. H., Nelson, J. A., Hoffman, I. F., Schock, J., Lamers, S. L., Goodman, M., Vernazza, P., Kazembe, P., Maida, M., Zimba, D., Goodenow, M. M., Eron, J. J., Jr., Fiscus, S. A., Cohen, M. S. & Swanstrom, R. (1999) J Virol 73, 6271-81.
10. Mosier, D. & Sieburg, H. (1994) Immunol Today 15, 332-9.
11. Kwong, P. D., Wyatt, R., Robinson, J., Sweet, R. W., Sodroski, J. & Hendrickson, W. A. (1998) Nature 393, 648-59. 25
12. Kwong, P. D., Wyatt, R., Majeed, S., Robinson, J., Sweet, R. W., Sodroski, J.& Hendrickson, W. A. (2000) Structure Fold Des 8, 1329-39.
13. Cordonnier, A., Montagnier, L. & Emerman, M. (1989) Nature 340, 5714.
14. Kowalski, M., Potz, J., Basiripour, L., Dorfman, T., Goh, W. C., Terwilliger, E., Dayton, A., Rosen, C., Haseltine, W. & Sodroski, J. (1987) Science 237, 1351-5. 30
15. Rizzuto, C. D., Wyatt, R., Hernandez-Ramos, N., Sun, Y., Kwong, P. D., Hendrickson, W. A. & Sodroski, J. (1998) Science 280, 1949-53.
16. Kraus, E., James, W. & Barclay, A. N. (1998) J Immunol 160, 5209-12.
17. Tahiri-Alaoui, A., Frigotto, L., Manville, N., Ibrahim, J., Romby, P. & James, W. (2002) Nucleic Acids Research 30, 1-9. 35
18. Sayer, N., Ibrahim, J., Turner, K., Tahiri-Alaoui, A. & James, W. (2002) Biochem Biophys Res Comm in press.
19. Gartner, S., Markovits, P., Markovitz, D. M., Betts, R. F. & Popovic, M. (1986) Jama 256, 2365-71.
20. Gendelman, H. E., Orenstein, J. M., Martin, M. A., Ferrua, C., Mitra, R., Phipps, T., Wahl, L. A., Lane, H. C., Fauci, A. S., Burke, D. S. & et al. (1988) J Exp Med 167, 142841.
21. Barre-Sinoussi, F., Chemann, J. C., Rey, F., Nugeyre, M. T., Chamaret, S., Gruest, J., Dauguet, C., Axler-Blin, C., Vezinet-Brun, F., Rouzioux, C., Rozenbaum, W. & Montagnier, L. (1983) Science 220, 868-71.
22. Sullivan, N., Sun, Y., Sattentau, Q., Thali, M., Wu, D., Denisova, G., Gershoni, J., Robinson, J., Moore, J. & Sodroski, J. (1998) J Virol 72, 4694-703.
23. Thali, M., Moore, J. P., Furman, C., Charles, M., Ho, D. D., Robinson, J. & Sodroski, J. (1993) J Virol 67, 3978-88.
24. Buchacher, A., Predl, R., Strutzenberger, K., Steinfellner, W., Trkola, A., Purtscher, M., Gruber, G., Tauer, C., Steindi, F., Jungbauer, A. & et al. (1994) AIDS Res Hum Retroviruses 10, 359-69.
25. Burton, D. R., Barbas, C. F., 3rd, Persson, M. A., Koenig, S., Chanock, R. M. & Lerner, R. A. (1991) Proc Natl Acad Sci USA 88, 10134-7.
26. Prince, A. M., Reesink, H., Pascual, D., Horowitz, B., Hewlett, I., Murthy, K. K., Cobb, K. E. & Eichberg, J. W. (1991) AIDS Res Hum Retroviruses 7, 971-3.
27. Gershoni, J. M., Denisova, G., Raviv, D., Smorodinsky, N. I. & Buyaner, D. (1993) Faseb J 7, 1185-7.
28. Lin, C. L., Sewell, A. K., Gao, G. F., Whelan, K. T., Phillips, R. E. & Austyn, J. M. (2000) J Exp Med 192, 587-94.
29. King, L. & Possee, R. (1992) The Baculovirus expression system: a laboratory guide (Chapman & Hall, London).
30. Karlsson, R., Michaelsson, A. & Mattsson, L. (1991) J Immunol Methods 145,229-40.
31. Collin, M., Herbein, G., Montaner, L. & Gordon, S. (1993) Res Virol 144, 13-9.
32. Moore, J. P., McKeating, J. A., Jones, I. M., Stephens, P. E., Clements, G., Thomson, S. & Weiss, R. A. (1990) Aids 4, 307-15.
33. Ellington, A. D. & Szostak, J. W. (1990) Nature 346, 818-22.
34. Fitzwater, T. & Polisky, B. (1996) Methods Enzymol 267, 275-301.
35. Pagratis, N. C., Bell, C., Chang, Y. F., Jennings, S., Fitzwater, T., Jellinek, D. & Dang, C. (1997) Nat Biotechnol 15, 68-73.
36. Pilgrim, A. K., Pantaleo, G., Cohen, 0. J., Fink, L. M., Zhou, J. Y., Zhou, J. T., Bolognesi, D. P., Fauci, A. S. & Montefiori, D. C. (1997) J Infect Dis 176, 924-32.
37. Moore, J. P. & Ho, D. D. (1995) Aids 9, S 17-36.
38. Corin, M., Illei, P., James, W. & Gordon, S. (1994) J Gen Virol 75, 1597-1603.
39. Borrow, P., Lewicki, H., Wei, X., Horwitz, M. S., Peffer, N., Meyers, H., Nelson, J. A., Gairin, J. E., Hahn, B. H., Oldstone, M. B. & Shaw, G. M. (1997) Nat Med 3, 205-11.
40. Trkola, A., Kuhmann, S. E., Strizki, J. M., Maxwell, E., Ketas, T., Morgan, T., Pugach, P., Xu, S., Wojcik, L., Tagat, J., Palani, A., Shapiro, S., Clader, J. W., McCombie, S., Reyes, G. R., Baroudy, B. M. & Moore, J. P. (2002) Proc Natl Acad Sci USA 99, 395-400.
41. Michael, N. L. & Moore, J. P. (1999) Nat Med 5, 740-2.
42. Wyatt, R., Moore, J., Accola, M., Desjardin, E., Robinson, J. & Sodroski, J. (1995) J Virol 69, 5723-33.
43. Wu, L., Gerard, N. P., Wyatt, R., Choe, H., Parolin, C., Ruffing, N., Borsetti, A., Cardoso, A. A., Desjardin, E., Newman, W., Gerard, C. & Sodroski, J. (1996) Nature 384, 179-83.
44. Zwick, M. B., Wang, M., Poignard, P., Stiegler, G., Katinger, H., Burton, D. R. & Parren, P. W. (2001) J Virol 75, 12198-208.
45. McKeating, J. A., Gow, J., Goudsmit, J., Pearl, L. H., Mulder, C. & Weiss, R. A. (1989) Aids 3,777-84.
46. Arendrup, M., Nielsen, C., Hansen, J. E., Pedersen, C., Mathiesen, L. & Nielsen, J. O. (1992) J Acquir Immune Defic Syndr 5, 303-7.
47. Kilby, J. M., Hopkins, S., Venetta, T. M., DiMassimo, B., Cloud, G. A., Lee, J. Y., Alldredge, L., Hunter, E., Lambert, D., Bolognesi, D., Matthews, T., Johnson, M. R., Nowak, M. A., Shaw, G. M. & Saag, M. S. (1998) Nat Med 4, 1302-7.
48. Ferrer, M., Kapoor, T. M., Strassmaier, T., Weissenhorn, W., Skehel, J. J., Oprian, D., Schreiber, S. L., Wiley, D. C. & Harrison, S. C. (1999) Nat Struct Biol 6, 953-60.
49. Bewley, C. A., Louis, J. M., Ghirlando, R. & Clore, G. M. (2002) J. Biol. Chem. 277, 14238-14245. 25
50. Chan, D. C., Chutkowski, C. T. & Kim, P. S. (1998) Proc Natl Acad Sci U S A 95, 15613-7.
51. Kilby, J. M., Hopkins, S., Venetta, T. M., DiMassimo, B., Cloud, G. A., Lee, J. Y., Alldredge, L., Hunter, E., Lambert, D., Bolognesi, D., Matthews, T., Johnson, M. R., Nowak, M. A., Shaw, G. M. & Saag, M. S. (1998) Nat Med 4, 1302-7.
52. Zollner, B., Feucht, H. H., Schroter, M., Schafer, P., Plettenberg, A., Stoehr, A. & Laufs, R. (2001) Aids 15, 935-6.

53. Rizzuto, C. & Sodroski, J. (2000) AIDS Res Hum Retroviruses 16, 741-9

TABLE 1

| | |
|---|---|
| B1 | CACCUACUAGACCACUUUUUGAGCCGGUUUUUUCGGG |
| | AACUUGCCAA |
| B3 | GACCGGUGUGUCCUGAUCCAACUGCCACAAGUACCAU |
| | AUGCAGGUGACGU |
| B4 | GAGCGGUUAAGGGAGAUUUAGGCAGCAGCUUGGACA |
| | GUGUAUCGGCUGAG |
| B5 | GGGCGCUUAAUGUAUGCCGUAUGACCCUCAACAUCCG |
| | ACUCAGUUAAGC |
| B9 | CCUCUUGCACCGCCGAGAAUAUAAUUCAAGAGGUCCA |
| | CAACUAAUUAG |
| B11 | CCAAGGGCUAAGUCCGCAAAUAUCCUUCCUAAAGGAC |
| | UCGUUACGUCGG |
| B19 | AGACCUUAUACCUGAGAUUACACGCUCUUCGAGCACG |
| | UCGAC |
| B28 | GCGAAAACUCCGAUUUUCCUCUGUAGUGAUGGGAUUU |
| | UCCCGCCUGAACC |
| B30 | CACCUACCUAAUUAUUAAACUUUGGGCAGUAUCCCGC |
| | UUUGCUUCUUAUC |
| B31 | GUUUAUAUAUACACAGGUUAAGCGUAACUUCGCUGGA |
| | CAGCAAGAAUCCU |
| B33 | CAUUGGCCAAUUCCUUGAAUCUCGACUGCUCGGUAGA |
| | AUAGA CCUUACCA |
| B36 | AGGAGAAUUAUGAGCGGGACAACUUCGUUCCGUGUUC |
| | GCGU ACUGAGCGC |
| B38 | CUUCUCCCUUGAGGGCCCCAUGACCUGACUGUAGAUA |
| | UCUGCCCUCGAG |
| B40 | UGUGGGCCACGCCCGAUUUUACGCUUUUACCCGCACG |
| | CGAUUGGUUUGUU |
| B44 | CAGUCGUCAUGGUUAUAGCUGCCACAACCUCGGUCCU |
| | GUCUUCAACGGCC |
| B45 | GUCAAGUGCACACCCUUGCUCGUUUCUCGAUCGCCAC |
| | AACCGAUUCCAAG |
| B55 | CUUGCCGUAGACCCAUUUUCCAAUCACAAGUCACGCG |
| | UCUCAAGCUGUUA |
| B62 | CCCGUACCACCACACCCUAUGCACAUCGUUGUUUGUC |
| | GUCUUUCCCGCAU |
| B81 | AGUUUCAUCGUCCGAGCAAGAUCCUAAUGGCGUCCGG |
| | CGCGUUUAUGACU |
| B82 | CCCCCAUGGCACGCCGAUCACGUUUUGCUGUCCGCCG |
| | GUCCAUAAAUACU |
| B84 | AUGACGUACCCGCACAAGCCACCACAAGUCUUAAUCG |
| | CGCCACCCUUGC |
| B86 | ACGUGCUCUCAUCUUUUAAUUCGUGGGCUCUGCGGCU |
| | AGCCUCUUAGCUC |
| B114 | CAUUACAGCGAAGUUACCAGCCAUACACGGUACAAAU |
| | GCGCCCGACUAGU |
| B116 | GACGGCAACCCGUUAUAACCUCCCACUGGCUAUCCCG |
| | UUAA GCUUCCCUA |
| B132 | UCACCUGUACACUACCUCUACCAUGCUUGAGCCUACG |
| | CCGC CGACACCC |
| B136 | CGUAUUCAUCAGOUAGCGUAGAUCCGUGUGGCGGGCU |
| | GUUC CAUUUUA |
| B137 | GCCAGGGUUCAUCAUUCACGGCCGAUUUCGAAGCUC |
| | CUAACUCGAGACAC. |

1. A nucleic acid molecule capable of binding to an envelope glycoprotein of an enveloped virus, wherein the binding of said nucleic acid molecule results in neutralizing said virus.

2. The nucleic acid molecule of claim 1, wherein said virus is HIV.

3. The nucleic acid molecule of or claim 2, wherein said virus is HIV-1.

4. The nucleic acid molecule claim 1, wherein said envelope glycoprotein is gp120.

5. The nucleic acid molecule of claim 1, wherein said nucleic acid molecule comprises the nucleic acid set forth in SEQ ID NO. 1, 2, 3, 4, 5, 6, 7, 8, 9, 10, 11, 12, 13, 14, 15, 16, 17, 18, 19, 20, 21, 22, 23, 24, 5, 26, 27 or combination thereof.

6. The nucleic acid molecule as of claim 1, wherein said nucleic acid molecule further comprises modified pyrimidine or purine nucleotide bases.

7. The modified nucleic acid molecule of claim 6, wherein said pyrimidine is modified in the 6 or 8 position.

8. A method for screening for potential therapeutic targets utilizing the nucleic acid molecule of claim 5.

9. The method of claim 8, wherein said method further comprising competitive inhibition.

10. A pharmaceutical composition comprising the nucleic acid molecule of claim 5.

11. A medicament for use in the treatment of HIV infection comprising the nucleic acid of claim 5.

12. A method for the treatment of HIV infection comprising administering an effective amount of the nucleic acid molecule as in claim 5 to a subject in need thereof.

13. The modified nucleic acid molecule of claim 6, wherein said purine is modified in the 5 position.

14. The modified nucleic acid molecule of claim 6, wherein said pyrimidine is modified in the 2 position.

15. The modified nucleic acid molecule of claim 13, wherein purine is modified in the 5 position with —CH₃, I, Br, or Cl

16. The modified nucleic acid molecule of claim 14, wherein pyrimidine is modified in the 2 position with —NH₃, O⁶—CH₃, N⁶—CH₃ or N²—CH₃.

17. The modified nucleic acid molecule of claim 14, wherein pyrimidine is modified in the 2 position with NH₂, F or OCH₃.

18. The pharmaceutical composition of claim 10, further comprising a pharmaceutically acceptable carrier, diluent or excipient.
